(19) United States
(12) Patent Application Publication
(10) Pub. No.: US 2021/0169985 A1
(43) Pub. Date: Jun. 10, 2021

(54) METHODS FOR TREATING GRAM POSITIVE BACTERIAL INFECTION

(71) Applicants: INSERM (Institut National de la Santé et de la Recherche Médicale), Paris (FR); Centre National de la Recherche Scientifique (CNRS), Paris (FR); Université de Paris, Paris (FR)

(72) Inventors: Carole PEYSSONNAUX, Paris (FR); Jacques MATHIEU, Paris (FR); Mariangela MALERBA, Paris (FR)

(21) Appl. No.: 17/052,383

(22) PCT Filed: May 4, 2018
(86) PCT No.: PCT/IB2018/000884

§ 371 Date: Nov. 2, 2020

(51) Int. Cl. A61K 38/22; A61P 31/04

(52) U.S. Cl. CPC A61K 38/22; A61P 31/04

(57) ABSTRACT

The present invention relies on the discovery that, surprisingly, when it is produced locally in the epidermis, hepcidin is able to directly initiate the recruitment of neutrophils by increasing the CXCL1 production in keratinocytes. While hepcidin had no direct antimicrobial activity against Group A Streptococcus (GAS), injection of hepcidin, at the site of infection prevented GAS systemic spread. Hepcidin agonists may represent a novel therapeutic to prevent life threatening bacteremia not only in streptococcal NF but also in complicated infections due to compromised host immunity. The present invention relates to a method for treating Gram-positive bacterial infection in a patient in need thereof, comprising administering to the patient hepcidin polypeptide. More specifically, it concerns method for treating with mature form of hepcidin, gram-positive bacterial infection such as Group A Streptococcus infection which could be associated with Necrotizing fasciitis (NF).

### FIELD OF THE INVENTION

The present invention relates to method for treating gram-positive bacterial infection with mature hepcidin. More specifically, it concerns method for treating with mature form of hepcidin gram-positive bacterial infection such as Group A Streptococcus infection associated with Necrotizing fasciitis (NF).

### BACKGROUND OF THE INVENTION

Necrotizing fasciitis (NF), also known as gangrene, was first described by Hippocrates in the 5th century BC¹. NF is characterized by widespread necrosis of the skin, subcutaneous tissues, and fascia. Even with optimal treatment, NF is associated with a fulminant course and high mortality rate, ranging from 25% to 35% in recent series². Group A Streptococcus (GAS), considered as the most common cause of necrotizing fasciitis, is associated with bacteremia and shock. The incidence of invasive S. pyogenes disease is increasing worldwide³. Upon detection of these Gram-positive pyogenic bacteria, the immune system launches a complex response which critically depends on the recruitment and activation of neutrophils⁴. Hepcidin is the key iron regulatory hormone is mainly produced by the hepatocytes. Inventors previously demonstrated that hepatic hepcidin is sufficient to ensure systemic iron homeostasis in physiological conditions suggesting that production of hepcidin by extra-hepatic tissues may have local roles⁵. Hepcidin first described as an antimicrobial peptide (AMP), but its putative expression and role in the infected skin, major source of AMPs have never been investigated.

Several recent studies (such Michels K R et al JCI Insight. 2017; 2(6):e92002 and Stefanova D. et al Blood. 2017 Jul. 20; 130(3):245-257.) show that hepcidin analogs (minihepcidins) can have an beneficial effect on Gram negative infection by limiting extracellular iron availability. But property of mature form of hepcidin on Gram-positive infection has never been described.

### SUMMARY OF THE INVENTION

Hepatic hepcidin is known as being the key hormone in iron homeostasis; it is able to decrease plasma iron levels by blocking iron absorption in the duodenum and iron release from macrophages thus targeting the two entrance gates for iron in the circulation. Several stimuli have been shown to be involved in hepcidin regulation: iron, hypoxia, erythropoietic demand and inflammation.

The present invention relies on the discovery that, surprisingly, when it is produced locally in the epidermis, mature form of hepcidin (hepc-25) is able to directly initiate the recruitment of neutrophils by increasing the CXCL1 production in keratinocytes. While hepcidin had no direct antimicrobial activity against GAS, injection of hepcidin, at the site of infection prevented GAS systemic spread. Hepcidin agonists may represent a novel therapeutic to prevent life-threatening bacteremia not only in streptococcal NF but also in complicated infections due compromised host immunity.

Accordingly, the present invention relates to a method of treating gram-positive bacterial infection in a patient in need thereof, comprising administering to the patient a polypeptide comprising a sequence of 20 amino acids having
at least 50% identity or at least 60% identity with the sequence SEQ ID NO: 1
cysteine residues at positions 2, 5, 6, 8, 9, 14, 17, and 18
   or of a nucleic acid encoding said polypeptide

More specifically, it concerns the discovery of the unexpected property of the hepcidin on CXCL1 production in keratinocytes, useful for the treatment of gram-positive bacterial infections and also useful to prevent (or treat) harmful consequence of such gram-positive bacterial infections, which could be associated with necrotizing fasciitis (NF).

### DETAILED DESCRIPTION OF THE INVENTION

In the present study, inventors demonstrate that expression of hepcidin was unexpectedly increased in the skin of streptococcal NF patients compared to healthy controls. To address its role, mice lacking hepcidin in keratinocytes (Hepc^{Δkerat}) were submitted to a GAS-induced necrotizing soft tissue infection model. Hepc^{Δkerat} mice failed to restrict systemic spread and showed a defect in neutrophil recruitment at the initial focus, as well as a decrease in the expression of CXCL1, a key neutrophil chemokine in the response to GAS. While hepcidin (hepc-25) had no direct antimicrobial activity against GAS, it unexpectedly increased the translation of CXCL1 in keratinocytes. Finally, injection of hepcidin, at the site of infection prevented GAS systemic spread.

The present invention relates to a method of treating gram-positive bacterial infection in a patient in need thereof, comprising administering to the patient a polypeptide comprising a sequence of 20 amino acids having
at least 50% identity or at least 60% identity with the sequence SEQ ID NO: 1
cysteine residues at positions 2, 5, 6, 8, 9, 14, 17, and 18
   or a nucleic acid encoding said polypeptide.

In particular embodiment of the invention, the Gram-positive bacteria infection is a skin Gram-positive bacteria infection.

Hepcidin was first identified as a liver-derived antimicrobial peptide. The human hepcidin gene encodes an 84-residue prepropeptide that contains a 24-residue N-terminal signal peptide that is subsequently cleaved to produce pro-hepcidin. Pro-hepcidin is then processed to produce a mature 25-amino acid hepcidin (Jordan et al., The Journal of Biological chemistry, 2009, 284, 36, 24155-24167).

It is now well established that hepatic hepcidin is the regulator of iron homeostasis (as disclosed by Lesbordes-Brion et al., Blood, 2006, 108, 1402-1405 and Nicolas et al., Proc. Natl. Acad. Sci. U.S.A., 2002, 99, 4596-4601). Hepcidin is a hypoferremic hormone; it binds to and degrades ferroportin (FPN, the only iron exporter known to date), thereby decreasing the iron levels in the circulation. Since its expression can be induced by inflammation (Nicolas et al., J. Clin. Invest., 2002, 110, 1037-1044), hepcidin has been proposed as an important mediator of Anemia of Chronic disease (ACD), also known as Anemia of Inflammation (AI).

Preferred polypeptides or nucleic acids for the method according to the invention are the mature forms of human hepcidin, represented for instance by a polypeptide of 20 amino-acids having the sequence

(SEQ ID NO: 1)
Ile Cys Ile Phe Cys Cys Gly Cys Cys His Arg Ser
Lys Cys Gly Met Cys Cys Lys Thr,

or by a polypeptide of 22 amino-acids having the sequence:

(SEQ ID NO: 2)
Phe Pro Ile Cys Ile Phe Cys Cys Gly Cys Cys His
Arg Ser Lys Cys Gly Met Cys Cys Lys Thr.

or by a polypeptide of 25 amino-acids having the sequence:

(SEQ ID NO: 3)
Asp Thr His Phe Pro Ile Cys Ile Phe Cys Cys Gly
Cys Cys His Arg Ser Lys Cys Gly Met Cys Cys Lys
Thr,

or nucleic acids encoding said polypeptides The term “hepcidin” should be understood broadly, it encompasses the mature forms of hepidin, variants and fragments thereof having same biological activity: increasing the CXCL1 production in keratinocytes.

Biological activity of mature form of hepcidin (CXCL1 production by keratinocyte) can be measured for example by supernatant cytokines quantification using Elisa (see experimental section and FIG. 2). or with the V-PLEX Proinflammatory Panel1 kit Meso Scale Discovery).

Precursors of said mature forms of hepcidin, i.e. prohepcidin and preprohepcidin and nucleic acids encoding said precursors can also be used.

Other examples of polypeptides or nucleic acids suitable for use according to the invention are vertebrate, preferably mammalian, homologous of mature forms of human hepcidin or precursors thereof, or nucleic acids encoding said polypeptides. Known vertebrate homologous of human hepcidin include for instance rat hepcidin, mouse hepcidin, trout hepcidin.

Preferably, if the patient is human, the hepcidin is a mature form of human hepcidin (UniProtKB-P81172). The protein sequence of said human hepcidin, may be found in NCBI database with the following access numbers: mRNA NM_NM_021175, and protein_id: NP_NP_066998 (hepcidin preproprotein)

Chimeric polypeptides, comprising the sequence of a mature form of hepcidin, can also be used.

The invention also encompasses the use of functional equivalents of the above-defined polypeptides. Functional equivalents are herein defined as peptide variants, having the same functional biological activity as the mature forms of hepcidin.

All these polypeptides and nucleic acids can be obtained by classical methods known in themselves. For instance, the 20 amino-acids and 25 amino-acids forms of hepcidin can be obtained from plasma or from urine, as disclosed by KRAUSE et al. (FEBS Left., 2000, vol. 480, 147-150); or PARK et al. (J. Biol. Chem., 2001, vol. 276, 7806-7810). Alternatively, they can be obtained by culturing cells expressing hepcidin, and recovering said polypeptide from the cell culture. According to a particular embodiment, said cells are host cells transformed by a nucleic acid encoding one of the polypeptides defined above.

Methods for producing hepcidin are well known. For example, hepcidin can be obtained by classical sequential procedures for hepcidin purification from total human plasma. Starting from human plasma, ADDO L et al (Int J Hematol. 2016 January; 103(1):34-43) purified 3 isoform of human hepcidin by using liquid chromatography-tandem mass spectrometry (LC-tandem MS). By using liquid chromatography coupled with tandem mass spectrometry (LC-MS/MS), a highly specific and quantitative serum hepcidin method (isoform 25) was also describe (Lefebvre T et al. Clin Chem Lab Med. 2015 Sep. 1; 53(10):1557-67 or Abbas et al, Anal Bioanal Chem. 2018 Apr. 18. doi: 10.1007/s00216-018-1056-0.). By using surface-enhanced laser-desorption/ionization time-of-flight mass spectrometry (SELDI-TOF MS), isoform of human hepcidin detection in serum and urine was also describe (Kemna et al., Clin Chem. 2007 April; 53(4):620-8.).

Alternatively, hepcidin can be a recombinant hepcidin as described in Gagliardo et al., FEBS J. 2008 August; 275(15):3793-803.

Chemical synthesis can also be used, in particular in the case of the peptide derivatives.

A nucleic acid encoding hepcidin can for instance be obtained from a genomic or cDNA library of a vertebrate, using suitable primers able to hybridize selectively with said nucleic acids. It can also be obtained by the classical techniques of polynucleotide synthesis.

Typically a variant of hepcidin has at least 50%, preferably, at least 60%, preferably, at least 70%, preferably, at least 80%, preferably, at least 85% more preferably at least 90%, more preferably at least 95% and even more preferably at least 99% identity with human mature form of hepcidin.

Typically, identity may be determined by BLAST or FASTA algorithms.

The term “gram-positive” bacterial infection refers to a local or systemic infection with gram-positive bacteria. Gram-positive bacteria are bacteria that give a positive result in the Gram stain test, which is traditionally used to quickly classify bacteria into two broad categories according to their cell wall. Gram-positive bacteria take up the crystal violet stain used in the test, and then appear to be purple-coloured when seen through a microscope. This is because the thick peptidoglycan layer in the bacterial cell wall retains the stain after it is washed away from the rest of the sample, in the decolorization stage of the test.

In the classical sense, six Gram-positive genera are typically pathogenic in humans. Two of these, Streptococcus and Staphylococcus, are cocci (sphere-shaped). The remaining organisms are bacilli (rod-shaped) and can be subdivided based on their ability to form spores. The non-spore formers are Corynebacterium and Listeria (a coccobacillus), whereas Bacillus and Clostridium produce spores (Gladwin, et al (2007). Miami, Fla.: MedMaster. pp. 4-5. ISBN 978-0-940780-81-1).

In particular embodiment of the invention the Gram-positive bacteria according to the invention are hyperinvasive bacteria

The term “hyperinvasive” for Gram-positive bacteria means that bacteria are resistant to innate immune clearance. Invasive bacterial infections occur when the bacteria invade the living tissue especially parts of the body where bacteria are not normally present (such as the bloodstream, soft tissues like muscle or fat, and the meninges) and evade the immune system of the person who is infected. This may occur when a person has sores or other breaks in the skin that allow the bacteria to get into the tissue, or when the person's ability to fight off the infection is decreased because of chronic illness or an illness that affects the immune system. Main example of Invasive Gram positive Bacterial Diseases are

### Streptococcal Disease, Group A Invasive or Streptococcal TSS

### Streptococcal Disease, Invasive Group B

Streptococcus pneumoniae (pneumococcus).

In particular embodiment of the invention, the Gram-positive bacteria according to the invention are selected from the group consisting of Streptococcus, Staphylococcus, Clostridium, Listeria, Bacillus and Corynebacterium.

In another particular embodiment of the invention, the Gram-positive bacteria according to the invention is Streptococcus selected from the group consisting of beta-hemolytic (Group A or Group B), alpha hemolytic (pneumonia) or gamma haemolytic (Enterococcus).

In more particular embodiment, the Gram-positive bacteria according to the invention is Group A beta-hemolytic Streptococcus (GAS).

In more particular embodiment, the Gram-positive bacteria according to the invention is a clone of hyperinvasive serogroup MITI group A beta-hemolytic Streptococcus (GAS).

Group A Streptococcus (GAS) also called “Streptococcus pyogenes” refers to specific species of Gram-positive bacteria. These bacteria are aerotolerant and an extracellular bacterium, made up of non-motile and non-sporing cocci. As expected with a streptococci, it is clinically important in human illness. It is an infrequent, but usually pathogenic, part of the skin microbiota. It is the predominant species harboring the Lancefield group A antigen, and is often called group A Streptococcus (GAS). Group A streptococci when grown on blood agar typically produces small zones of beta-hemolysis, a complete destruction of red blood cells. (A zone size of 2-3 mm is typical.) It is thus also called group A (beta-hemolytic) Streptococcus (GABHS), and can make colonies greater than 5 mm in size.

An estimated 700 million GAS infections occur worldwide each year. While the overall mortality rate for these infections is 0.1%, over 650,000 of the cases are severe and invasive, and have a mortality rate of 25% (Aziz R K, et al (2010). PLoS ONE. 5 (4): e9798). Early recognition and treatment are critical; diagnostic failure can result in sepsis and death.

As described in the experimental data (FIG. 2F), the treatment with mature form of hepcidin of gram-positive infection, such as Group A Streptococcus (GAS) infection allows to prevent (or to treat) harmful consequence of gram positive bacterial infection such as Necrotizing fasciitis (NF).

Accordingly, the invention also provides a method of treating gram-positive bacterial infection associated with Necrotizing fasciitis (NF) in a patient in need thereof with mature form of hepcidin according to the invention.

As used herein, the term “patient” denotes a mammal, such as a rodent, a feline, a canine, and a primate. Preferably, a patient according to the invention is a human.

In a particular embodiment of the invention, said patient is suffering from gram-positive bacterial infections. In a more particular embodiment of the invention, said patient is suffering from a skin gram-positive bacterial infection.

In a specific embodiment, the patient is at risk to develop a gram positive bacterial infections, such as a patient before a medical or surgical intervention, very young and very old subject (infants and seniors), people with chronic or serious illnesses (including diabetes and cancer), and immunocompromised patients.

In a specific embodiment, very young subject (infant), means subject with less than 5, 4, 3, 2, 1 years old.

In a specific embodiment, very old subject (senior), means subject more than, 70, 71, 72, 73, 74, 75, 76, 77, 78, 79, 80; 81; 82; 83; 84; 85; 86; 87; 88; 89; 90; 91; 92; 93; 94; 95; 96; 97; 98; 99 or 100 years old.

The present invention also relates to a method for treating gram-positive infection in a patient at risk to develop such infection, such method involving the step of administering to a patient in need thereof a therapeutically effective amount of mature form of hepcidin. This gram-positive bacterial infection could be associated with Necrotizing fasciitis (NF).

In particular embodiment of the invention, the Gram-positive bacterial infection is a skin Gram-positive bacterial infection.

By a “therapeutically effective amount” is meant a sufficient amount to be effective, at a reasonable benefit/risk ratio applicable to any medical treatment. It will be understood, however, that the total daily usage will be decided by the attending physician within the scope of sound medical judgment. The specific therapeutically effective dose level for any particular patient in need thereof will depend upon a variety of factors including the age, body weight, general health, sex and diet of the patient, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

The hepcidin can be administered in a suitable formulation to humans and animals by topical or systemic administration, including oral, rectal, nasal, buccal, ocular, sublingual, transdermal, rectal, topical, vaginal, parenteral (including subcutaneous, intra-arterial, intramuscular, intravenous, intradermal, intrathecal and epidural), intracisternal and intraperitoneal. It will be appreciated that the preferred route may vary with for example the condition of the recipient. In a preferred embodiment hepcidin is administered by parenteral way (including subcutaneous, intra-arterial, intramuscular, intravenous, intradermal, intrathecal and epidural).

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]) Typically medicaments according to the invention comprise a pharmaceutically-acceptable carrier. A person skilled in the art will be aware of suitable carriers. Suitable formulations for administration by any desired route may be prepared by standard methods, for example by reference to well-known text such as Remington; The Science and Practice of Pharmacy.

### FIGURE LEGENDS

FIG. 1. (A) Generation of Hepc^{Δkerat} mice. Recombination of the floxed hamp1 allele by genomic PCR in the epidermis of the Hepc^{Δkerat14} and WT mice. (B) Hepcidin deletion in keratinocytes renders mice more susceptible to GAS infection. Bacterial count in skin, blood and spleen of WT vs Hepc^{Δkerat14} mice four days after injection with GAS. At least seven mice in each group were tested in three independent experiments. Statistical analysis was performed using unpaired Student's t-test; *p<0.05. ** p<0.01.

FIG. 2—

(A-C) Hepcidin promotes CXCL1 production at the translational level. CXCL1 levels measured by ELISA in the culture supernatant of murine primary keratinocytes stimulated for 1 or 3 hours with hepcidin vs PBS. (B) Expression by Q-PCR of CXCL1 in WT primary keratinocyte incubated for 1 and 3 hours with 360 nM, 3.6 μM hepcidin or PBS. n>4; mean+/−s.e.m. (C) CXCL1 levels measured by ELISA in the culture supernatant of murine primary keratinocytes stimulated for with 3.6 μM hepcidin in presence of solvent/actinomycinD/cycloheximide/brefeldinA. Statistical analysis was performed using unpaired Student's t-test; * p<0.05. (D) Area of necrotic ulcers in skin of WT and Hepc^{Δkerat14} mice four days after injection with GAS. (E) Bacterial count in the skin of WT and Hepc^{Δkerat14} mice +/− injected daily with CXCL1 four days after injection with GAS. (F) Local injection of hepcidin prevents systemic spread of infection. Weight variation in infected mice treated with 3 subcutaneous injection of hepcidin (circle) or PBS (square) during 96 hours (left). GAS dissemination in the spleen of GAS-infected mice 96 hours after infection (right). Ten mice in each group were tested in two experiments. Statistical analysis was performed using unpaired Student's t-test; *p<0.05. ** p<0.01.

FIG. 3. Hepcidin has no antimicrobial activity against GAS in vitro and does not influence keratinocyte antimicrobial activity. (A) GAS growth curve in presence of penicillin G, LL-37 and hepcidin. (B) GAS killing kinetics of 32 μM of LL-37 and hepcidin. (C) Bacterial recovering at 2 h and 4 h following incubation of log-phase GAS with murine primary keratinocytes from WT and Hamp1Δ^{ker} mice. Data are representative of two independent experiments preformed in triplicate. Statistical analysis was performed using unpaired Student's t-test; ** p<0.01.

FIG. 4 Mini-hepcidin does bot promote CXCL1 production in keratinocytes. CXCL1 levels measured by ELISA in the culture supernatant of murine primary keratinocytes stimulated for 1 or 3 hours with mini-hepcidin or PBS.

### EXAMPLE

### Materials

### Mice

Hamp1^{lox/lox} mice⁵ were bred with K14-Cre transgenic mice (Jonkers et al., 2001 Nat Genet. 2001; 29:418-425), in which the Cre recombinase is under the control of the murine K14 promoter. Studies were performed in a C57BL6/genetic background, using 8-12 week old male littermates.

The animal studies described here were reviewed and approved (Agreement no CEEA34.CP.003.13) by the “President du Comité d'Ethique pour l'Expérimentation Animale Paris Descartes” and are in accordance with the principles and guidelines established by the European Convention for the Protection of Laboratory Animals (Council of Europe, ETS 123, 1991).

### Mouse Model of GAS Infection

A well-established model of necrotizing soft tissue infection was adapted for this study (⁷,⁹). In brief, 100 μl of a mid logarithmic growth phase (approximately 10⁷ c.f.u.) of GAS was mixed with an equal volume of sterile Cytodex beads (Sigma-Aldrich) and injected subcutaneously into the flank of 8- to 12-week-old WT and Hepc^{Δkerat14} male littermates, previously shaved (veet hair removal cream). Weight loss and lesion size of mice were daily estimated. After 96 hours mice were sacrificed, skin necrotic ulcers, spleen, and blood (via retro orbital bleeding) were recovered and homogenized in 1:2 mg/μl of sterile PBS. Serial dilutions of tissue homogenate were stretched on THA plates, in order to enumerate living c.f.u.

For rescue experiment WT and Hepc^{Δkerat} littermates were subcutaneously infected with 10⁷ c.f.u. of GAS with mock or 1 μg/mouse of rCXCL1 (R&D system, 1395-KC-025/CF). Mice were intralesionally injected again at 24, 48 and 72 h with mock or 1 μg of rCXCL1.

For therapeutic treatment C57BL6 mice were infected as described above. One day after infection, animals were subcutaneously injected with PBS (control) or 1 μg of hepcidin (peptide international). Mice were treated again at 48 and 72 hours with 500 ng of hepc-25. After 96 hours mice were sacrificed, spleens were taken, homogenized for c.f.u. enumeration.

### Human Patients

Informed consent to the protocol was obtained for all subjects. Biopsies were fixed in 10% formalin and embedded in paraffin.

### Isolation and Treatment of Murine Primary Keratinocytes.

Keratinocytes were isolated from the skin of 2-day-old WT or Hepc^{Δkerat} mice, based on modifications from a previously described protocol⁸. In brief, dermis were easily separated from epidermis after floating newborn mice skin on freshly thawed cold trypsin 0.25% (thermo fisher 15050065) overnight at 4° C. with the dermis side down. Cells were isolated in stop trypsin medium (DMEM, high glucose, no glutamine, no calcium (thermo fisher 21068028) containing 20% heat inactivated and Chelex (Bio rad 1421253) treated—FBS, 1% GlutaMAXSupplement (thermo fisher 35050061), 1% Sodium Pyruvate (thermo fisher 11360070), 100 U/ml Penicillin, 100 μg/ml Streptomycin (thermo fisher), 2.5 μg/ml Amphotericin B (Gibco)).

Keratinocytes were obtained by epidermis mechanical dissociation through shaking at 350 rpm for 1 hour at 37° C. The resulting cell suspension was filtered through 100 μm filter, centrifuged at 250 g for 10 min at 4° C., plated on fibronectin and bovine collagen I coated tissue culture plate and incubated at 37° C. in a humidified 5% CO2 atmosphere. The cells were cultured in 0.07 mM calcium medium (low Ca²⁺) containing EGF (10 ng/ml) and cholera toxin (10⁻¹⁰ mol/L). Once confluence reached, cells were switched in 1.2 mM calcium medium (high Ca²⁺) in order to assure their differentiation. After three days in high Ca′ cells were differentiated and suitable for different experiments.

### Reverse Transcription and Real-Time Quantitative PCR

RNA extraction, reverse transcription and quantitative PCR have been performed as previously described {Zumerle, 2014 #46}. All samples were normalized to the threshold cycle value for cyclophilin-A.

### Quantification of Cytokines

Keratinocytes, previously starved of FBS for 1 hour, were treated with 1 or 10 μg/ml hepcidin (Peptide International) and incubated for different time points. In some experiments, inhibitors were added 30 minutes before hepcidin treatment during FBS starving: brefaldin A (sigma B7651), cycloheximide (sigma C7698), actinomycinD (sigma A1410). The cells were lysed with Tri reagent (sigma) while supernatant cytokines were measured with the V-PLEX Proinflammatory Panel1 kit (Meso Scale Discovery), according to the manufacturer's instruction.

### Antimicrobial Assays

As previously described, 9 GAS NZ131 were grown in Todd Hewitt Broth (THB, Sigma) plus 0.3% yeast extract to early log phase (absorbance at 600 nm, A600=0,2). Bacteria were then diluted in THB to A600=0,001 (≈2×105 colony forming units (c.f.u.) m1-1). This bacterial suspension was kept growing overnight at 37° C. in presence of PBS, LL-37, hepcidin (peptide international) or penicillin G (sigma P7794). For antimicrobial killing kinetics, exponential phase GAS (A600=0,4) was exposed to PBS or LL-37 or hepcidin and incubated at 37° C. At different time point serial dilutions of these bacterial suspensions were plated on THA plate for counting surviving c.f.u.

### Keratinocyte Killing Assay

Keratinocytes from WT and Hepc^{Δkerat} newborn mice were isolated and cultured as above described. Their antimicrobial function was tested with log-phase GAS culture.

Cells were previously starved of antibiotics for 2 hours then infected with a multiplicity of infection (MOI) of 10 bacteria per cell. GAS were centrifuged onto keratinocytes at 350 g for 10 min, in order to assure bacteria-cell interaction. Infected cells were incubated at 37° C./5% CO2 for different time points. At each time point, well contents were harvested and spread on THA plates for enumerating surviving c.f.u.

### Results

We examined hepcidin expression, by immunohistochemical staining, in the skin of healthy subjects and patients suffering from streptococcal necrotizing fasciitis (NF) (detailed in table 1). Hepcidin immunoreactivity was increased in NF suffering patients compared to healthy controls. To investigate the role of hepcidin in the etiology of NF, we chose an established animal infection model of GAS induced necrotizing soft tissue infection^{6,7}. The GAS inoculum was introduced subcutaneously (SC) into a shaved area on the flank of WT mice. In skin biopsies of the infected lesions, hepcidin was not only expressed in myeloid cells recruited to the site of infection, as previously reported¹⁰ but was also strongly detected in keratinocytes.

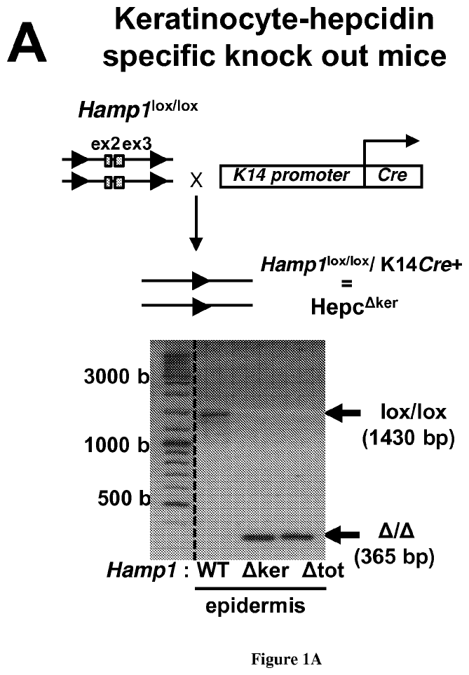

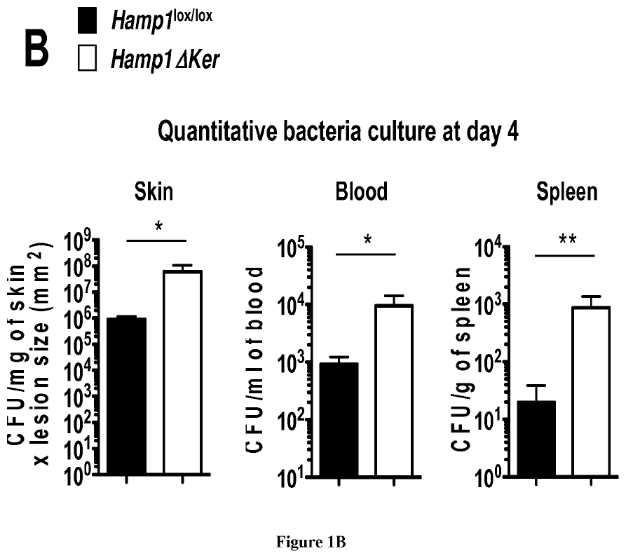

To probe the functional significance of keratinocyte-derived hepcidin in vivo, we developed a novel mouse model of keratinocyte-specific (Hepc^{Δkerat}) by crossing Hepc^{lox/lox} mice with K14^{cre} mice. We observed an efficient truncation of the floxed hamp1 allele in the epidermis of the Hepc^{Δkerat} mice, with deletion of the hamp1 allele in the total hepcidin KO mice as a positive control (FIG. 1A). Hepc^{lox/lox} and Hepc^{Δkerat} mice were submitted to the GAS induced necrotizing soft tissue infection model. Quantitative bacterial cultures were performed 4 days post-infection in the skin ulcer, blood, and spleen. Hepc^{Δkerat} mice had a significantly higher number of bacteria than the WT littermates at the lesion site (6.10⁷ vs 9.10⁵ CFU/mg) but also in the blood (10⁴ vs 9.10² CFU/ml) and in peripheral organs such as the spleen (9.10² vs 20 CFU/mg) indicating that keratinocyte production of hepcidin was important in limiting the ability of GAS to replicate within the necrotic skin tissues and to disseminate from the initial focus of infection into the bloodstream and systemic organs.

To investigate the mechanisms by which epidermal hepcidin restrains the infection, we first determined the bacteriostatic and bactericidal effects of hepcidin against GAS. The activity of hepcidin was evaluated by its capacity to inhibit GAS growth as compared to LL-37. Hepcidin had no direct bacteriostatic effect against GAS in vitro, while, at the same concentration, the antimicrobial peptide LL-37 was able to prevent the growth of the bacteria (FIG. 3A). The bactericidal activity was assessed by adding hepcidin to the culture medium at 4 μg/ml. While bactericidal activity corresponded to a significant reduction in bacterial titers after LL-37 exposure as compared to control, addition of hepcidin had no effect (FIG. 3B). Moreover, primary keratinocytes derived from WT and Hepc Δ^{kerat} mice displayed the same bactericidal activity against this pathogen (FIG. 3C).

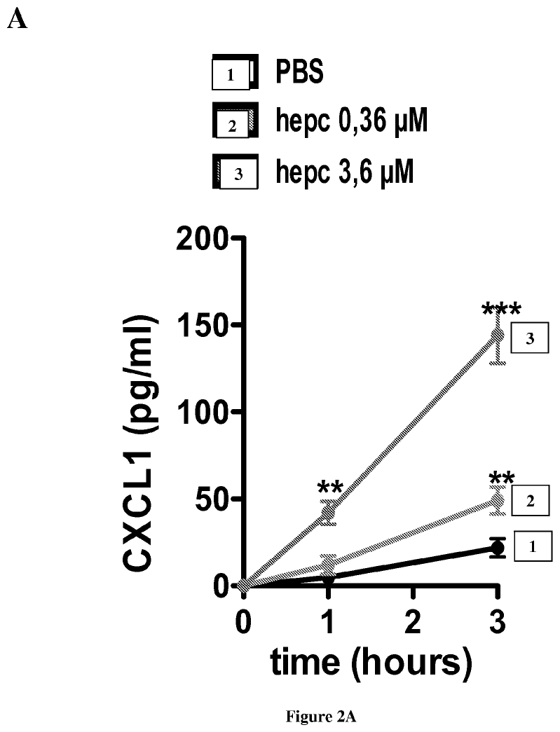

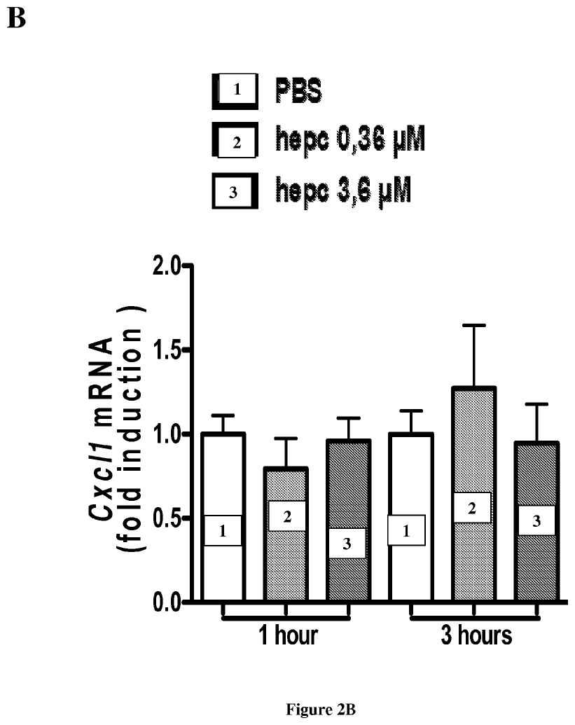

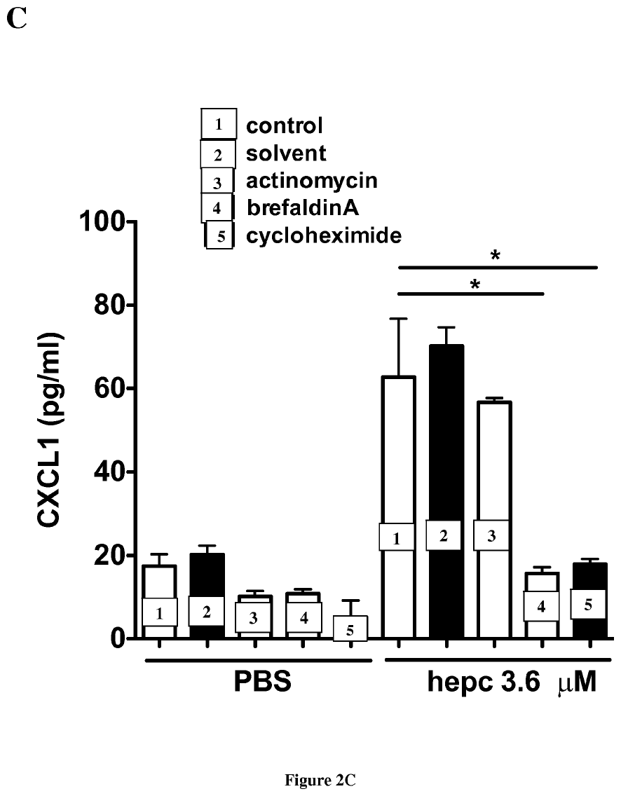

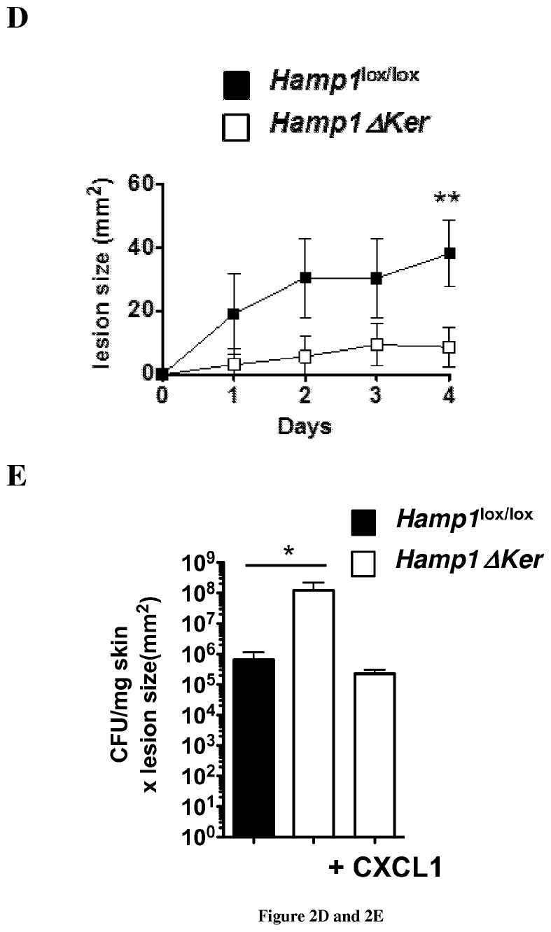

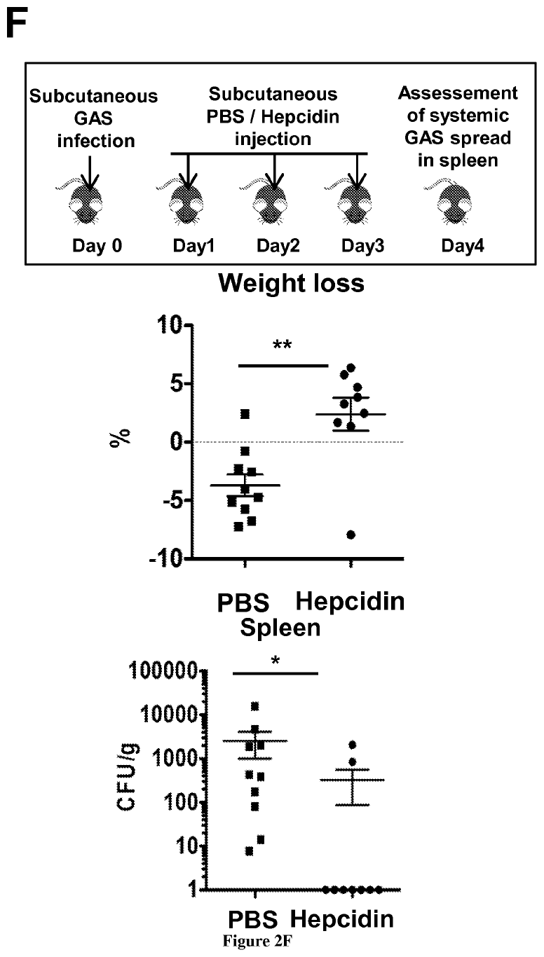

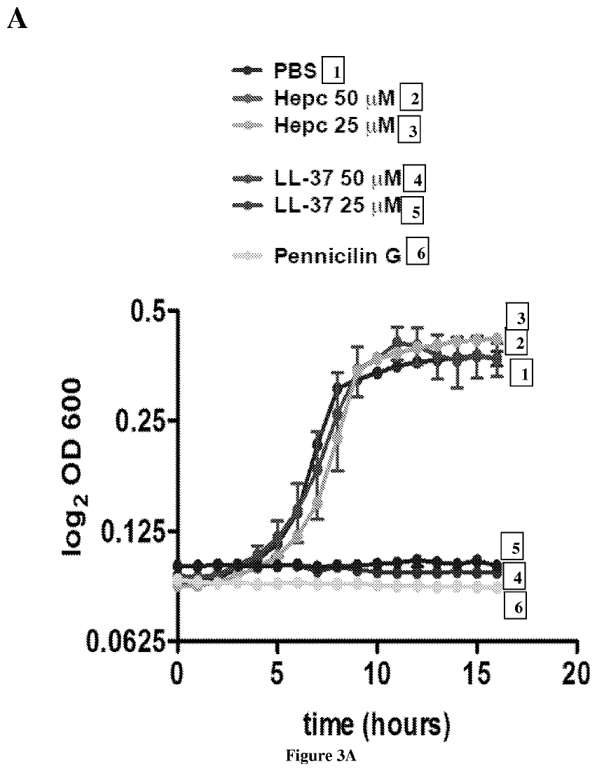

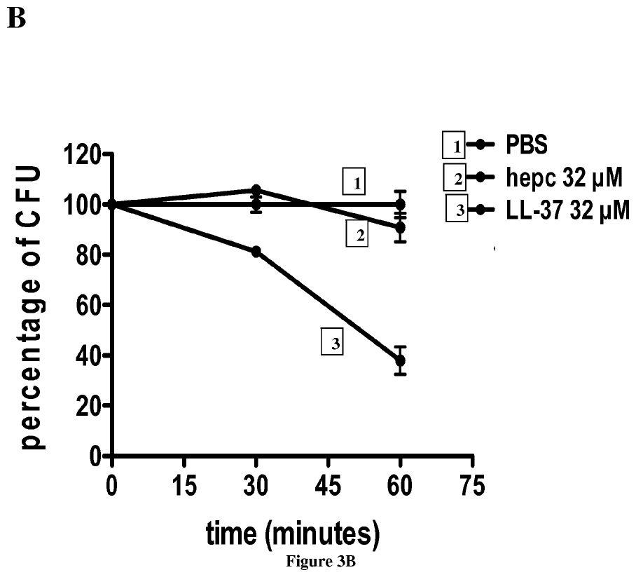

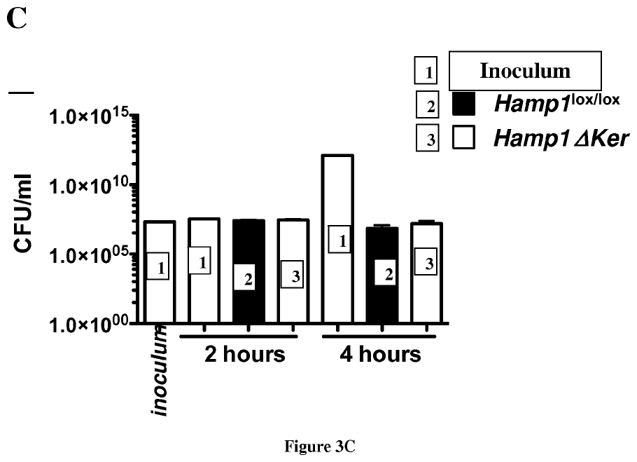

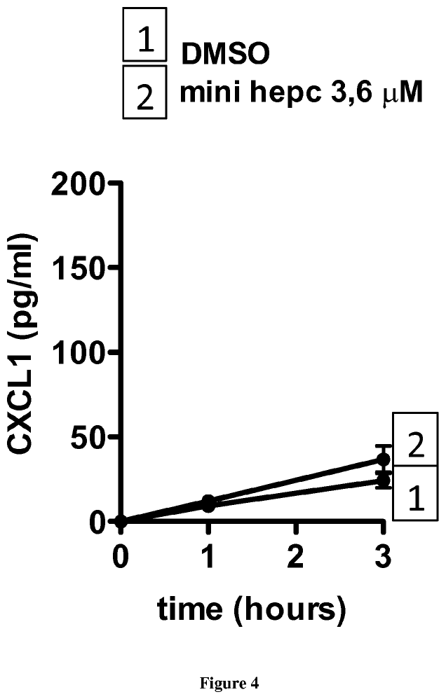

We therefore asked whether hepcidin could have a direct immunomodulatory role on keratinocytes. A cytoplex was performed on murine primary keratinocytes, incubated with 360 nM and 3.6 μM hepcidin. Hepcidin induced a dose dependent increase of CXCL1 production (FIG. 2A) but not of other tested inflammatory cytokines. CXCL1 levels in the cell culture supernatant reached respectively 150 and 50 pg/ml at 3 hours at the dose of 360 nM and 3.6 μM (FIG. 2A). Intriguingly, hepcidin, did not increase CXCL1 expression at the mRNA level. (FIG. 2B). Cycloheximide, but not actinomycin D, significantly reduced hepcidin-induced CXCL1 production, suggesting a possible role for hepcidin on CXCL1 mRNA translation (FIG. 2C). In contrast, a murine mini-hepcidin {Preza, 2011 #180}, consisting in the 9 N-terminal amino acids of hepcidin was not able to trigger CXCL1 production. These data show that, while mini-hepcidin is sufficient to degrade ferroportin and regulate iron homeostasis, it is not sufficient to display this new immunomodulary role (FIG. 4).

In corroboration with the in vitro results, the expression of CXCL1 was decreased in the keratinocytes of GAS infected-Hepc^{Δkerat} mice compared to WT littermates, showing that keratinocyte-derived hepcidin contributes to CXCL1 production in vivo.

As a consequence of the decrease in CXCL1 production, a significant decrease in neutrophil recruitment was observed in the skin of Hepc^{Δkerat} mice as compared to the control littermates. This defect in keratinocyte-derived hepcidin to recruit neutrophils at the site of infection was translated in a decrease in the necrotic skin lesion size of the Hepc^{Δkerat} mice as compared to controls (FIG. 2D). Subcutaneous injection of CXCL1 to Hepc^{Δkerat} mice decreased the number of bacteria at the level of WT mice, confirming that the lack of CXCL1 production was responsible for the susceptibility of mutant mice to infection.

PMNs fulfill a key role in preventing the transition of GAS soft tissue infections from local to rapidly disseminating life-threatening diseases⁴. We asked whether injection of hepcidin could have a therapeutic action in the GAS induced necrotizing soft tissue infection model. 24 hours after GAS infection, 1 μg of hepcidin/mock was subcutaneously injected at the inoculation site, followed by two injections of 500 ng of hepcidin for 2 consecutive days (FIG. 2F). At the end of the treatment, the weight was measured as a marker of morbidity. In contrast to mock-injected mice, that exhibited weight loss (FIG. 2F) and signs of morbidity such as rough hair coat, hunched posture (data not shown), hepcidin-treated mice didn't present any signs of morbidity and did not loose weight. Remarkably, whereas all the control mice presented with systemic bacterial dissemination (as shown by the number of bacteria in the spleen), 7 out of 9 hepcidin-treated mice showed no dissemination (FIG. 2F).

The standard treatment of NF consists of broad-spectrum antibiotics, wide surgical debridement, and supportive care. Even with optimal treatment, NF portend significant morbidity and have high mortality rates². Hepcidin agonists may represent a novel therapeutic to prevent life-threatening bacteremia not only in streptococcal NF but also in complicated infections due to compromised host immunity.

TABLE 1

| Patient | Age | | Underlying | Preceeding | Type of | | |
|---|---|---|---|---|---|---|---|
| No. | (y) | Site | disease | trauma | infection | Sepsis | Outcome |
| 1 | 57 | Leg | None | No | Group A | No | Survived |
| | | | | | Streptococcus | | |
| 2 | 51 | Leg | None | Yes | Group A | Yes | Survived |
| | | | | | Streptococcus | | |
| 3 | 80 | Leg | Prostate | No | Streptococcus | Yes | Died |
| | | | adenocarcima | | dysgalactiae | | |
| 4 | 27 | Thumb | None | Yes | Group A | No | Survived |
| | | | | | Streptococcus | | |
| 5 | 72 | Leg | None | No | Group A | Yes | Survived |
| | | | | | Streptococcus | | |
| | | | | | MRSA | | |

### REFERENCES

Throughout this application, various references describe the state of the art to which the invention pertains. The disclosures of these references are hereby incorporated by reference into the present disclosure.
1. Descamps, V., Aitken, J. & Lee, M. G. Hippocrates on necrotising fasciitis. Lancet 344, 556 (1994).
2. Hakkarainen, T. W., Kopari, N. M., Pham, T. N. & Evans, H. L. Necrotizing soft tissue infections: review and current concepts in treatment, systems of care, and outcomes. Curr Probl Surg 51, 344-362 (2014).
3. Sims Sanyahumbi, A., Colquhoun, S., Wyber, R. & Carapetis, J. R. Global Disease Burden of Group A Streptococcus. in Streptococcus pyogenes: Basic Biology to Clinical Manifestations (eds. Ferretti, J. J., Stevens, D. L. & Fischetti, V. A.) (Oklahoma City (Okla.), 2016).
4. Walker, M. J., et al. Disease manifestations and pathogenic mechanisms of Group A Streptococcus. Clin Microbiol Rev 27, 264-301 (2014).
5. Zumerle, S., et al. Targeted disruption of hepcidin in the liver recapitulates the hemochromatotic phenotype. Blood 123, 3646-3650 (2014).
6. Datta, V., et al. Mutational analysis of the group A streptococcal operon encoding streptolysin S and its virulence role in invasive infection. Mol Microbiol 56, 681-695 (2005).
7. Peyssonnaux, C., et al. HIF-1alpha expression regulates the bactericidal capacity of phagocytes. J Clin Invest 115, 1806-1815 (2005).
8. Lichti, U., Anders, J. & Yuspa, S. H. Isolation and short-term culture of primary keratinocytes, hair follicle populations and dermal cells from newborn mice and keratinocytes from adult mice for in vitro analysis and for grafting to immunodeficient mice. Nat Protoc 3, 799-810 (2008).
9. Nizet, V., et al. Innate antimicrobial peptide protects the skin from invasive bacterial infection. Nature 414, 454-457 (2001).
10. Peyssonnaux, C. et al. TLR4-dependent hepcidin expression by myeloid cells in response to bacterial pathogens. Blood (2006). doi:10.1182/blood-2005-06-2259

1. Method for treating a Gram positive bacterial infection in a patient in need thereof, comprising administering to the patient
a polypeptide comprising a sequence of 20 amino acids having
at least 50% identity or at least 60% identity with the sequence SEQ ID NO: 1, and
cysteine residues at positions 2, 5, 6, 8, 9, 14, 17, and 18;
or
a nucleic acid encoding said polypeptide.

2. The method according to claim 1, wherein said polypeptide is a vertebrate homologue of a mature form of human hepcidin.

3. The method according to claim 2 wherein said vertebrate homologue is a mammalian homologue.

4. The method according to claim 1, wherein the polypeptide is a polypeptide of 25 amino-acids having the sequence SEQ ID NO: 3.

5. The method according to claim 1, wherein the Gram positive bacterial infection is caused by a gram positive bacteria that is a hyperinvasive bacteria.

6. The method according to claim 1, wherein the gram positive bacteria is selected from the group consisting of Streptococcus, Staphylococcus, Clostridium, Listeria, Bacillus and Corynebacterium.

7. The method according to claim 6 wherein the gram positive bacteria is a Streptococcus selected from the group consisting of a beta-hemolytic Streptococcus, an alpha hemolytic Streptococcus and a gamma haemolytic Streptococcus.

8. The method according to claim 7 wherein the gram positive bacteria is a Group A beta-hemolytic Streptococcus (GAS).

9. The method according to claim 1, wherein the Gram positive bacterial infection is associated with Necrotizing fasciitis (NF).

10. The method of claim 7, wherein the beta-hemolytic Streptococcus is a Group B beta-hemolytic Streptococcus.


SEQUENCE LISTING (3 sequences)

SEQ ID NO: 1 - protein, 20 aa, Homo sapiens
ICIFCCGCCHRSKCGMCCKT

SEQ ID NO: 2 - protein, 22 aa, Homo sapiens
FPICIFCCGCCHRSKCGMCCKT

SEQ ID NO: 3 - protein, 25 aa, Homo sapiens
DTHFPICIFCCGCCHRSKCGMCCKT